(19) United States
(12) Patent Application Publication
(10) Pub. No.: US 2008/0069734 A1
(43) Pub. Date: Mar. 20, 2008

(54) Patterned deposition using compressed carbon dioxide

(76) Inventors: Bushra Al-Duri, Birmingham (GB); Filipe Gaspar, Oeiras (PT); Andrew Bruce Holmes, Parkville (AU); Wilhelm Theodorus Stefanus Huck, Cambridge (GB); Gary Leeke, Worcester (GB); Tiejun Lu, Birmingham (GB); Christine Keiko Luscombe, Seattle, WA (US); Jonathan Seville, Birmingham (GB); Regina Santos, Birmingham (GB)

Correspondence Address: Fisher Technology Law, 40452 Hickory Ridge Place, Aldie, VA, 20105, US

(21) Appl. No.: 11/891,485

(22) Filed: Aug. 10, 2007

Related U.S. Application Data
(62) Division of application No. 10/433,780, filed on Oct. 28, 2003.

(30) Foreign Application Priority Data
Dec. 6, 2000 (GB) 0029673.1
Jun. 26, 2001 (GB) 0115538.1
Dec. 6, 2001 (GB) PCT/GB01/05402

(51) Int. Cl. H01L 29/12; C12Q 1/68; H01L 21/02

(52) U.S. Cl. 422/99; 257/40; 438/500; 257/E21.002

(57) ABSTRACT

A method for the patterned deposition of a material comprises the steps of dissolving or suspending said material in a solvent phase comprising compressed carbon dioxide, and depositing the solution or suspension onto a surface, evaporation of the solvent phase leaving a patterned deposit of said material. This method is particularly suitable for the patterned deposition of polymers and small organic molecules in organic light emitting diodes and organic transistors.

This patent application is a divisional patent application of U.S. Ser. No. 10/433,780, filed Oct. 28, 2003, which claims the priority benefit of United Kingdom Patent Application No. 0029673.1, filed Dec. 6, 2000, and United Kingdom Patent Application No. 0115538.1 filed Jun. 26, 2001, which are incorporated herein by reference.

### FIELD OF THE INVENTION

The present invention relates to a method for the patterned deposition of materials such as polymers and small organic molecules on surfaces, and to devices manufactured using this process.

### BACKGROUND TO THE INVENTION

Supercritical carbon dioxide has been used for polymer synthesis and polymer processing. This has been extensively reviewed in the past and the state of the art is summarised in an article by Cooper [A. I. Cooper, J. Mater. Chem., 2000, 10, 207]. Compressed carbon dioxide is also used as a solvent for the preparation of organic molecules and this has been summarised in a special issue of Chemical Reviews. [see Special Issue: Chem. Rev. 1999, 99, #2]. Unlike conventional liquid solvents, carbon dioxide is highly compressible and the density (and therefore solvent properties) can be tuned over a wide range by varying the pressure [see M. McHugh et al. “Supercritical Fluid Extraction” Boston, Butterworth-Heinemann, 1994]. Compressed carbon dioxide is a superior solvent medium for heavily fluorinated compounds. Compressed carbon dioxide has also been used as a (co-)solvent in the preparation of finely feathered sprays to coat surfaces with polymers [D. C. Busby, et al., Surf. Coat. Int 1991 74, 362; J. M. DeSimone, et al., J. Supercrit. Fluids 1999, 15, 173]. These prior art feathered sprays have been used to coat the surfaces of stone in the walls of historical buildings and the like with a protective layer of fluropolymer.

The use of supercritical fluids for the production of particles has increased enormously within the past few years due to their readily adjustable densities and particularly their pressure-dependent solvent power. In the RESS-process (rapid expansion of supercritical solutions) the expansion of a supercritical solution leads to a decrease in solvent power and hence to the precipitation of the organic or inorganic solute [J. W. Tom and P. G. Debenedetti, J. Aerosol Sci., 1991, 22, 555]. As the decompression is fast, high supersaturation is reached and fine powders can be obtained. The most common solvent in this process is carbon dioxide because of its mild critical temperature (31° C.) and its relatively low critical pressure (73 bar). As a result of the mild temperature, the RESS-process offers the opportunity to micronise heat-sensitive organics. These are otherwise difficult to comminute as they are thermally degraded. Another advantage of carbon dioxide is that it is a gas under atmospheric conditions so that the end-reaction mixture is solvent-free.

The morphology of the precipitated product is strongly influenced by the conditions of the expansion process. This is due to variations in nozzle design, solution concentration and parameters such as pressure and temperature [F. E. Hénon, M. Camaiti, A. L. C. Burke, R. G. Carbonell, J. M. DeSimone and F. Piacenti, J. Supercrit. Fluid., 1999, 15, 173].

In recent years, there has been considerable interest in light emitting organic materials such as conjugated polymers. Light emitting polymers possess a delocalised pi-electron system along the polymer backbone. The delocalised pi-electron system confers semiconducting properties to the polymer and gives it the ability to support positive and negative charge carriers with high mobilities along the polymer chain. Thin films of these conjugated polymers can be used in the preparation of optical devices such as light-emitting devices. These devices have numerous advantages over devices prepared using conventional semiconducting materials, including the possibility of wide area displays, low dc working voltages and simplicity of manufacture. Devices of this type are described in, for example, WO-A-90/13148, U.S. Pat. No. 5,512,654 and WO-A-95/06400.

The world market for displays based on organic and polymeric light-emitting materials has recently been estimated by Stanford Resources, Inc., to be $200 million in the year 2002 with a strong growth rate which fuels the high industrial interest in this area (D. E. Mentley, “Flat Information Displays: Market and Technology Trends”, 9^{th} edition, 1998). Efficient and highly stable LED devices with low power consumption, which fulfill commercial requirements, have been prepared by a number of companies and academic research groups (see, for example, R. H. Friend et al., Nature 1999, 397, 12).

At the moment, great efforts are dedicated to the realization of a full-colour, all plastic screen. The major challenges to achieve this goal are: (1) access to conjugated polymers emitting light of the three basic colours red, green and blue; (2) the conjugated polymers must be easy to process and fabricate into full-colour display structures. Organic electroluminescent devices such as polymeric LEDs (PLEDS) show great promise in meeting the first requirement, since manipulation of the emission colour can be achieved by changing the chemical structure of the organic emissive compound. However, while modulation of the chemical nature of the emissive layer is often easy and inexpensive on the lab scale it can be an expensive and complicated process on the industrial scale.

The second requirement of the easy processability and build-up of full-colour matrix devices raises the question of how to micro-pattern fine multicolour pixels and how to achieve full-colour emission. Inkjet printing and hybrid inkjet printing technology have recently attracted much interest for the patterning of organic LED devices (see, for example, R. F. Service, Science 1998, 279, 1135; Wudl et al., Appl. Phys. Lett. 1998, 73, 2561; J. Bharathan, Y. Yang, Appl. Phys. Lett. 1998, 72, 2660; and T. R. Hebner, C. C. Wu, D. Marcy, M. L. Lu, J. Sturm, Appl. Phys. Lett. 1998, 72, 519). Many problems exist in adapting this technology for the patterning of organic LED devices, not least being that of finding suitable solvents for the polymers and small organic molecules to enable them to be sprayed in a controlled manner onto the surface of the devices. It would be highly desirable to find a solvent which can be used for many of the typical small organic molecules and polymers used in organic LED devices which enables precise patterning of the devices with small droplets of the polymer solution or small organic molecule solution, and which is inexpensive, non-toxic, non-flammable, dissolves the polymers at mild temperatures and is a gas under atmospheric conditions.

Organic field-effect transistors (FETs) have recently become of interest for applications in cheap, logic circuits integrated on plastic substrates [C. Drury, et al., APL 73, 108 (1998)] and optoelectronic integrated devices and pixel transistor switches in high-resolution active-matrix displays [H. Sirringhaus, et al., Science 280, 1741 (1998), A. Dodabalapur, et al. Appl. Phys. Lett. 73, 142 (1998)]. In these FETs, the semiconducting layer comprises an organic semiconducting material. These organic semiconductors include both polymers and small organic molecules. Examples of suitable polymers include (optionally substituted) polythiophenes such as polythiophene and poly(3-alkylthiophenes) [Horowitz, Advanced Materials, 10, 365, (1998)]; and copolymers which incorporate thiophene and fluorene units such as poly-9,9′dioctyl-fluorene-co-bithiophene (F8T2) [H. Sirringhaus, et al., Applied Physics Letters, 77, 406 (2000)]. Examples of suitable small organic molecules include oligothiophenes (typically containing 3 to 6 thiophene units), pentacenes and phthalocyanines.

In test device configurations with a polymer semiconductor and inorganic metal electrodes and gate dielectric layers high-performance FETs have been demonstrated. Charge carrier mobilities up to 0.1 cm²/Vs and ON-OFF current ratios of 10⁶˜10⁸ have been reached which is comparable to the performance of amorphous silicon FETs [H. Sirringhaus, et al., Advances in Solid State Physics 39, 101 (1999)].

Typical architecture of polymer FETs is shown in Horowitz, Advanced Materials, 10, 365, (1998).

Thin, device-quality films of conjugated polymer semiconductors can be formed by coating a solution of the polymer in an organic solvent onto the substrate. The technology is therefore ideally suited for cheap, large-area solution processing compatible with flexible, plastic substrates. In particular, it enables processing using high-resolution inkjet printing techniques to produce thin-film transistor circuits (Physics Today, February 2001). However, as for the organic LEDs, various problems exist with the adoption of inkjet technology, including the choice of solvent.

Other applications in which the precise patterning of polymers, small organic molecules and organic and inorganic particles onto surfaces is required include the patterning of DNA and protein molecules in arrays (e.g. in DNA chips, probes and biosensors) and in catalyst arrays.

### SUMMARY OF THE INVENTION

It is an object of the present invention to provide a method for the patterned deposition of materials onto surfaces in a controlled manner using a cheap, environmentally friendly solvent.

It is a further object of the present invention to provide devices incorporating patterned materials deposited using the method of the present invention.

Thus, in a first aspect of the present invention, there is provided a method for the patterned deposition of a material comprising the steps of:

(i) dissolving or suspending said material in a solvent phase comprising compressed carbon dioxide; and

(ii) depositing the solution or suspension onto a surface, evaporation of the solvent phase leaving a patterned deposit of said material.

By compressed carbon dioxide, we mean carbon dioxide which has been compressed under pressure to produce liquid carbon dioxide or supercritical carbon dioxide. Compressed carbon dioxide has excellent properties that enables it to dissolve or have suspended therein a wide range of materials such as small organic molecules, polymers and inorganic or organic particles, these solutions and suspensions being ideal for the patterned deposition of the dissolved or suspended materials.

By patterned deposition we mean any means of creating a pattern on a surface including the directing of sprays or droplets of the material dissolved or suspended in compressed carbon dioxide directly onto a surface so as to create the pattern directly on said surface or by the directing of sprays or droplets of the material dissolved or suspended in compressed carbon dioxide onto patterned surfaces created by chemical and physical modification including surfaces modified by a lithographic technique or a printing technique.

Small organic molecules, polymers and inorganic or organic particles (ranging, for example, in size from 1 nm to 1 μm) can be deposited from compressed carbon dioxide.

Preferably, deposition of the material from carbon dioxide onto the surface involves the use of a device designed for the purpose, said device usually incorporating a capillary tube or nozzle from which the material is deposited. One preferred means of deposition employs a spray coating device to deliver feathered sprays of solutions or suspensions of the materials mentioned above. Another, most preferred option for deposition employs a modified version of an inkjet printer (e.g. such as the modified inkjet printers disclosed in R. F. Service, Science 1998, 279, 1135; Wudl et al., Appl. Phys. Lett. 1998, 73, 2561; J. Bharathan, Y. Yang, Appl. Phys. Lett. 1998, 72, 2660; T. R. Hebner, C. C. Wu, D. Marcy, M. L. Lu, J. Sturm, Appl. Phys. Lett. 1998, 72, 519; WO-A-01/16251; and Physics Today, February 2001, the contents of said publications being incorporated herein by reference thereto).

Thus, in a preferred embodiment of the present invention, there is provided a method for the patterned deposition of a material comprising supplying a solution processible formulation via a plurality of elongate bores onto a surface, wherein said formulation comprises said material dissolved or suspended in a solvent phase comprising compressed carbon dioxide.

Examples of particularly preferred embodiments of the material to be dissolved or suspended in the compressed carbon dioxide include organic light emitting materials such as a light emitting small organic molecules and light emitting conjugated polymers and organic semiconducting materials such as semiconducting small organic molecules and semiconducting polymers.

Small amounts of co-solvents such as water, trifluoroethanol, acetone, ethanol or methanol may be added to aid in the formation of a homogeneous solution or suspension, as well as in the formation of a fine spray. Furthermore, in some instances small amounts of surfactants such as perfluorinated polyethers (e.g. Fomblin) can be added to aid the formation of a homogeneous solution or suspension.

The morphology of the precipitated material is strongly influenced by the conditions of the expansion process. This can be affected by variations in nozzle design, solution concentration and parameters such as pressure and temperature.

In the modified inkjet process, droplets of the solution are deposited via a plurality of elongate tubes. The tubes are very narrow in diameter, typically having an internal diameter in the region of 20 to 200 μm. The formation of the droplet can be influenced by optimising the residence time between the gas and the liquid phases and the ratio of the length to the diameter of the tubes.

Compressed carbon dioxide is an attractive solvent for the patterned deposition of materials because it is inexpensive, non-toxic, and non-flammable. The use of carbon dioxide as a solvent for patterned deposition is of major environmental as well as economic importance. The use of carbon dioxide in controlled deposition onto surfaces has clear applications in many techniques which involve the patterned deposition of polymers, small organic molecules and the like, such applications including desktop publishing and fabrication of optical and electronic devices.

One particularly preferred application of this technique for the patterned deposition of materials is in the deposition of organic and polymeric materials for optoelectronic devices including organic and polymer LEDs and transistors and associated circuitry.

One of the current issues in passive matrix driven display devices is to address individual columns and rows with the avoidance of crosstalk. Alternatively these issues can be resolved by the use of thin film transistors to provide active matrix addressing. These issues have been fully discussed in articles by J. H. Burroughes, D. D. C. Bradley, A. R. Brown, R. N. Marks, K. MacKay, R. H. Friend, P. L. Burn and A. B. Holmes, Nature, 1990, 347, 539; A. Kraft, A. C. Grimsdale and A. B. Holmes, Angew. Chem. Int. Ed. Engl., 1998, 37, 402;, R. H. Friend, R. W. Gymer, A. B. Holmes, J. H. Burroughes, R. N. Marks, C. Taliani, D. D. C. Bradley, D. A. dos Santos, J. L. Brédas, M. Lögdlund and W. R. Salaneck, Nature, 1999, 397, 121; H. Sirringhaus, T. Kawase, R. H. Friend, T. Shimoda, M. Inbasekaran, W. Wu, and E. P. Woo, Science, 2000, 290, 2123.

The unique properties of compressed carbon dioxide should provide particular advantages in its use as a solvent in the deposition of organic and polymer layers in organic and polymer LEDs and transistors in terms of the resolution of the patterns produced and in the controlled evaporation of the processing solvent to afford high quality thin films.

Of the materials currently used for the organic light emitting layer in organic LEDs, many of the small organic molecules such as aluminium quinolinol complexes (Alq₃ complexes: see, for example U.S. Pat. No. 4,539,507), complexes of transition metals, lanthanides and actinides with organic ligands such as TMHD (see WO-A-00/26323) and quinacridone, rubrene and styryl dyes (see, for example, JP-A-264692/1988), the contents of which references are incorporated herein by reference thereto, should be easily soluble in compressed carbon dioxide. This makes them particularly suitable for adoption in the patterned deposition method of the present invention, thus enabling the simple, cheap and controlled patterning of films of these compounds in the construction of organic LEDs. On the other hand, most of the polymers currently used in organic LEDs such as poly-phenylene-vinylene (PPV) and derivatives thereof (see, for example, WO-A-90/13148), polyfluorene derivatives (see, for example, A. W. Grice, D. D. C. Bradley, M. T. Bernius, M. Inbasekaran, W. W. Wu, and E. P. Woo, Appl. Phys. Lett. 1998, 73, 629, WO-A-00/55927 and Bernius et al., Adv. Materials, 2000, 12, No. 23, 1737), polynaphthylene derivatives, polyindenofluorene derivatives and polyphenanthrenyl derivatives, the contents of which references are incorporated herein by reference thereto, are insoluble in compressed carbon dioxide. However, these polymers are easily derivatised and it should be relatively easy to introduce groups into these polymers which increase their solubility in compressed carbon dioxide, thus making them amenable to the method of patterned deposition of the present invention. (e.g. see “Polymer synthesis and processing using supercritical carbon dioxide” by A. I. Cooper, J. Mater. Chem., 2000, 10,207-234. Typical features which endow solubility to polymers in compressed carbon dioxide include the presence of perfluorinated or partially fluorinated alkyl and aryl substituents, polysiloxanes and specialist features such as those present in the polyether/polycarbonate described by T. Sarbu, T. Styranec and E. Beckman, Nature, 2000, 405, 165).

At their most basic, organic electroluminescent devices generally comprise an organic light emitting material which is positioned between a hole injecting electrode and an electron injecting electrode. The hole injecting electrode (anode) is typically a transparent tin-doped indium oxide (ITO)-coated glass substrate. Zirconium-doped indium oxide (Applied Physics Letters, 78 (8) 1050 (2001), Kim, H et al) and aluminium-doped zinc oxide (Applied Physics Letters, 76 (3) 259 (2000), Kim H et al) films have also been used as the anode. The material commonly used for the electron injecting electrode (cathode) is a low work function metal such as calcium or aluminium.

The choice of anode material for organic electroluminescent devices is based on several criteria: the anode must have good optical transparency, good electrical conductivity, good chemical stability and, preferably, a work function that lies near the HOMO levels of the organic materials to which it will inject holes. Doped indium oxide largely meets these criteria. Alternatives as the anode material that have also been tried include: titanium nitride [Advanced Materials, 11 (9) 727 (1999), Adamovich V, et al.]; high work function transparent conducting oxides including Ga—In—Sn—O and Zn—In—Sn—O [Advanced Materials, 13 (19) 1476 (2001), Cui, J., et al]; polymeric materials such as polystyrenesulfonic acid-doped polyaniline [Applied Physics Letters, 70 (16) 2067 (1997), Carter S. A et al, and Applied Physics Letters, 64 (10) 1245 (1994) Yang Y et al.].

The organic light emitting layer can comprise mixtures or discrete layers of two or more different emissive organic materials.

Typical device architecture is disclosed in, for example, WO-A-90/13148; U.S. Pat. No. 5,512,654; WO-A-95/06400; R. F. Service, Science 1998, 279, 1135; Wudl et al., Appl. Phys. Lett. 1998, 73, 2561; J. Bharathan, Y. Yang, Appl. Phys. Lett. 1998, 72, 2660; T. R. Hebner, C. C. Wu, D. Marcy, M. L. Lu, J. Sturm, Appl. Phys. Lett. 1998, 72, 519); and WO 99/48160; the contents of which references are incorporated herein by reference thereto.

The injection of holes from the hole injecting layer such as ITO into the organic emissive layer is controlled by the energy difference between the hole injecting layer work function and the highest occupied molecular orbital (HOMO) of the emissive material, and the chemical interaction at the interface between the hole injecting layer and the emissive layer. The deposition of high work function organic materials on the hole injecting layer, such as poly(styrene sulfonate)-doped poly (3,4-ethylene dioxythiophene) (PEDOT/PSS), N,N′-diphenyl-N,N′-(2-naphthyl)-(1,1′-phenyl)-4,4′-diamine (NBP) and N,N′-bis(3-methylphenyl)-1,1′-biphenyl-4,4′-diamine (TPD), provides “hole transport” layers which facilitate the hole injection into the light emitting layer, transport holes stably from the hole injecting electrode and obstruct electrons. These layers are effective in increasing the number of holes introduced into the light emitting layer.

In a further embodiment of the present invention there is provided a method for the manufacture of an organic light emitting diode comprising dissolving or suspending an emissive small organic molecule or an emissive conjugated polymer in a solvent phase comprising compressed carbon dioxide, and then depositing the solution or suspension onto the desired layer of said light emitting diode, evaporation of the solvent phase leaving a patterned light emitting layer comprising the deposited emissive small organic molecule or emissive conjugated polymer. In a particularly preferred embodiment, the solution or suspension of the emissive small organic molecule or emissive conjugated polymer is deposited via a plurality of elongate bores. The present invention also provides an organic light emitting diode manufactured according to this method.

Another application of the method of the present invention is the patterned deposition of the organic semiconducting layers in organic transistors. These organic semiconductors include both polymers and small organic molecules. The small organic molecules currently used, which include oligothiophenes (typically containing 3 to 6 thiophene units), pentacenes and phthalocyanines, should be soluble in compressed carbon dioxide, making them particularly suitable for adoption in the patterned deposition method of the present invention, thus enabling the simple, cheap and controlled patterning of films of these compounds in the construction of polymeric transistors. Again, however, most of the polymers currently used [e.g. (optionally substituted) polythiophenes such as polythiophene and poly(3-alkylthiophenes) [Horowitz, Advanced Materials, 10, 365, (1998)]; and copolymers which incorporate thiophene and fluorene units such as poly-9,9′dioctyl-fluorene-co-bithiophene (F8T2) [H. Sirringhaus, et al., Applied Physics Letters, 77, 406 (2000)], the contents of which are incorporated herein by reference thereto] are insoluble in compressed carbon dioxide. However, these polymers are also easily derivatised so that it should be relatively easy to introduce moieties which will enable their solubilisation in compressed carbon dioxide (e.g. as for the emissive organic polymers above).

Typical architecture of polymer transistors is shown in Horowitz, Advanced Materials, 10, 365, (1998), the contents of which are incorporated herein by reference thereto.

In a further embodiment of the present invention there is provided a method for the manufacture of an organic transistor comprising dissolving or suspending a semiconducting small organic molecule or a semiconducting conjugated polymer in a solvent phase comprising compressed carbon dioxide, and then depositing the solution or suspension onto the desired layer of said organic transistor, evaporation of the solvent phase leaving a patterned semiconducting layer comprising the deposited semiconducting small organic molecule or semiconducting conjugated polymer. In a particularly preferred embodiment, the solution or suspension of the semiconducting small organic molecule or semiconducting conjugated polymer is deposited via a plurality of elongate bores. The present invention also provides an organic transistor manufactured according to this method.

The method of the present invention for the use of compressed carbon dioxide in the patterned deposition of materials also has other applications such as the deposition into array format of catalysts, DNA, or proteins or materials for sensors. The method of the present invention can also be used in the manufacture of organic solid state photovoltaic devices in which the microphase separation of materials of different charge density, which could be deposited by the method of the invention, determines the ability to promote charge separation (see, for example, Halls et al, Nature, 1995, 376, 498).

In one example, we have found that a perfluorinated polymer [poly(methyl methacrylate) carrying a perfluorinated alkyl side chain] can be dissolved in liquid carbon dioxide. The polymer solution was deposited by use of a nozzle onto a pre-patterned surface. The resulting polymer film showed a clear image of the original pattern as measured by optical microscopy.

The straightforward integration of multiple organic layers (for the fabrication of red, green, and blue emitters for colour displays) would require the patterning of the individual organic layers. Because of their lack of solubility in and sensitivity to aqueous solutions and many solvents, the patterning of such organic materials by conventional photoresist and wet processing techniques is difficult. Effort to date to integrate organic light emitting diodes (OLEDs) which emit different colours on the same substrate have patterned them only indirectly (through the use of cathodes evaporated through shadow mask as dry-etch masks), or avoided the issue all together by putting the three devices on top of each other (relying on shadow masks to pattern the organics so that contacts to the multiple layers may be made). Since some of the conducting (or conjugated) polymers and many of the small organic polymers are solution processable, inkjet printing technology is an ideal method for printing polymer and small organic light-emitting diodes with high resolution. The use of compressed carbon dioxide, with all the advantages described above, provides a particularly favourable technique for high resolution patterning of the organic films.

### BRIEF DESCRIPTION OF THE DRAWINGS

The present invention may be further understood by consideration of the following embodiments of the present invention, with reference to the following drawings in which:

FIG. 1 shows a schematic diagram of a method for spraying or dropping a polymer onto a silicon substrate;

FIGS. 2 and 3 are photographs showing the effect of spraying compressed carbon dioxide solutions of a fluorinated polymer on pre-patterned silicon substrates;

FIG. 4 is a photograph showing the effect of spraying compressed carbon dioxide solutions of a fluorinated polymer via a capillary tube on pre-patterned silicon substrates; and

FIG. 5 is a photograph of one of the pre-patterned substrates.

### DESCRIPTION OF PREFERRED EMBODIMENTS

### EXAMPLE 1

### Fine Sprays

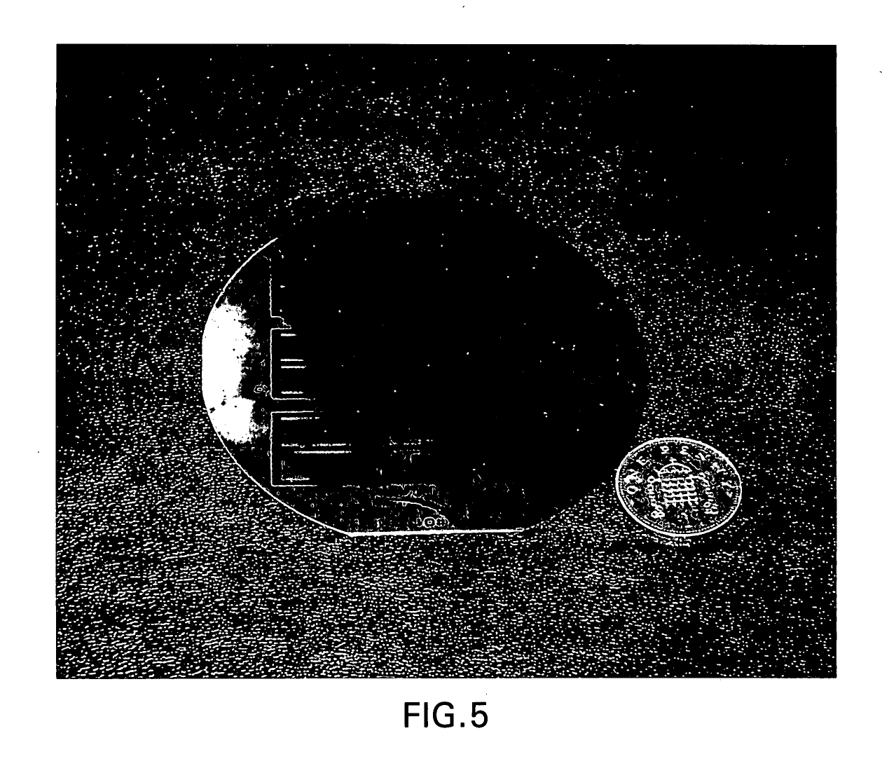

2 mg of either poly(perfluorooctyl methacrylate) (PFOMA) or poly(perfluoro-octyl acrylate) (PFOA) were placed inside a stainless steel vessel suitable for storing compressed carbon dioxide and adjusting the temperature and pressure thereof, such as that described, for example, by Hems, et al. (J. Mater. Chem., 1999, 9, 1403). The vessel was initially filled with liquid C0₂ at a temperature of 20° C. and a pressure of 103 bar (1500 psi). The polymer dissolved in the liquid CO₂ to form a colourless solution. In further experiments, 0.05 ml of water and methanol respectively were included as co-solvents and the perfluorinated polyether Fomblin® (0.05 ml) was included as a surfactant. The cell was then vented via a narrow metal tube to give a feathered spray of the polymer solution while holding a patterned silicon wafer approximately 10 cm away from the nozzle. The wafer (supplied by Mr Masaya Ishida of Seiko Epson) was patterned by lithography to give hydrophilic and hydrophobic areas, the PFOA and PFOMA sticking to the hydrophilic areas to give the patter A photograph of such a wafer is shown in FIG. 5. The wafer was then studied under the microscope and revealed an identical polymer pattern to that on the original surface of the wafer, thus proving the excellent potential that compressed carbon dioxide has as a solvent for use in the deposition of polymeric materials via fine sprays.

Results of spraying experiments with CO₂

| Polymer | Cosolvent | Solubility | Results |
|---|---|---|---|
| PFOMA^{a} | | CO₂ | Yes |
| PFOA^{b} | Water + Fomblin^{c} | CO₂ | Yes |
| PFOMA^{a} | Methanol + Fomblin | CO₂ | Yes |

^{a}Synthesised by anionic polymerisation.
^{b}PFOA = poly(perfluoro-octyl acrylate)
^{c}Trade mark of Ausimont.

### EXAMPLE 2

### Deposition by Spraying via a Modified Micrometering Valve

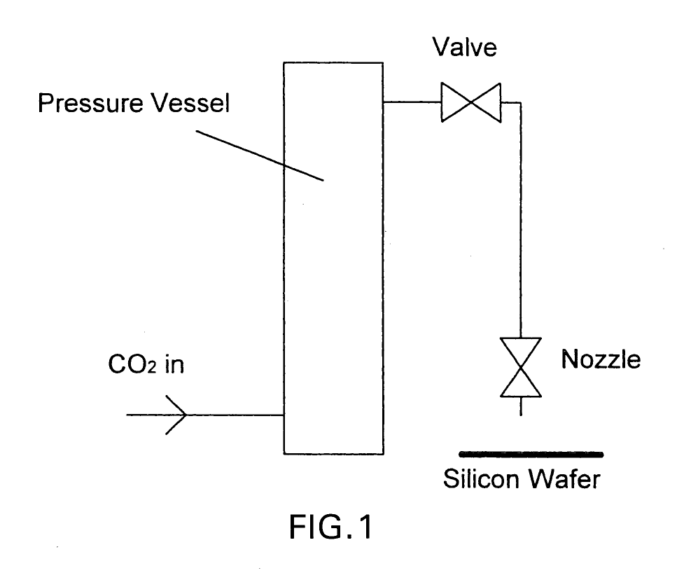

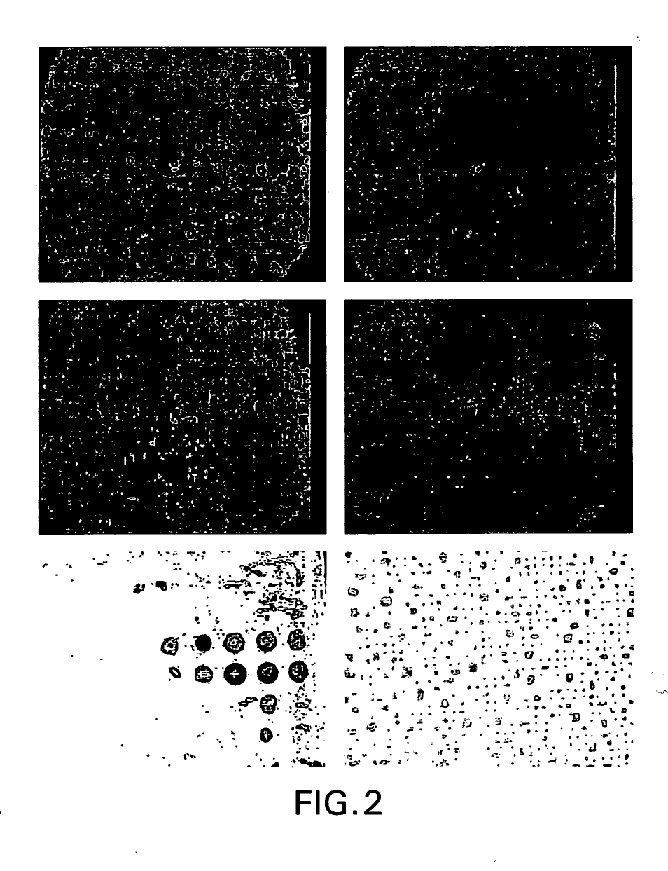

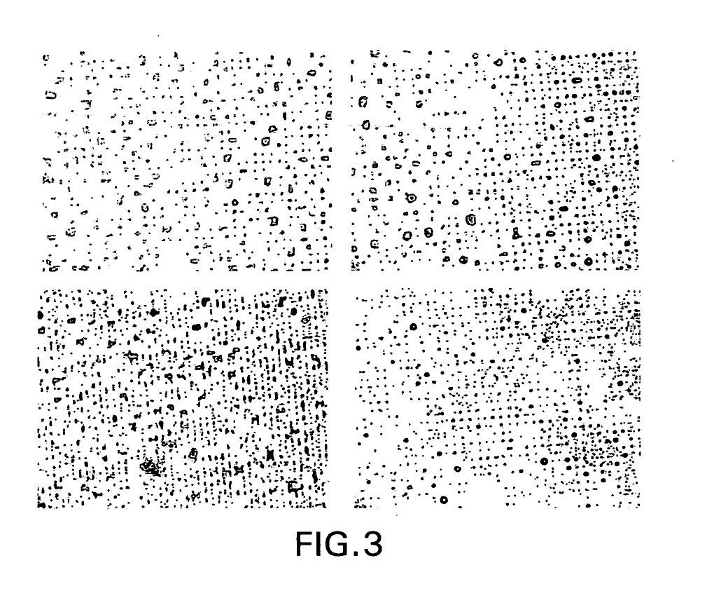

0.5 g of Fluorolink® (a perfluorinated polyether manufacture by Ausimont) were placed inside a stainless steel pressure vessel suitable for storing compressed carbon dioxide and adjusting the temperature and pressure thereof, such as that described, for example, by Hems, et al. (J. Mater. Chem., 1999, 9, 1403). A schematic representation of the vessel is shown in FIG. 1. The vessel was initially filled with liquid CO₂ and the temperature adjusted to room temperature and the pressure to 100 bar. The polymer dissolved in the liquid CO₂ to form a colourless solution. The cell was then vented via a nozzle which was a modified micrometering valve to give a feathered spray of the polymer solution while holding a patterned silicon wafer approximately 10 cm away from the nozzle. The flow-rate of the solution was between 1 and 5 kg h⁻¹ and the residence time of the wafers in the spraying stream was between 30 and 60 seconds. The wafer (supplied by Mr Masaya Ishida of Seiko Epson) was patterned by lithography to give hydrophilic and hydrophobic areas, the polymer sticking to the hydrophobic areas. The wafer was then studied under the microscope. Photographs of the patterns obtained are shown in FIGS. 2 and 3. The photographs show an identical polymer pattern to that on the original surface of the wafer.

### EXAMPLE 3

### Deposition by Dropping via Capillary Tubes

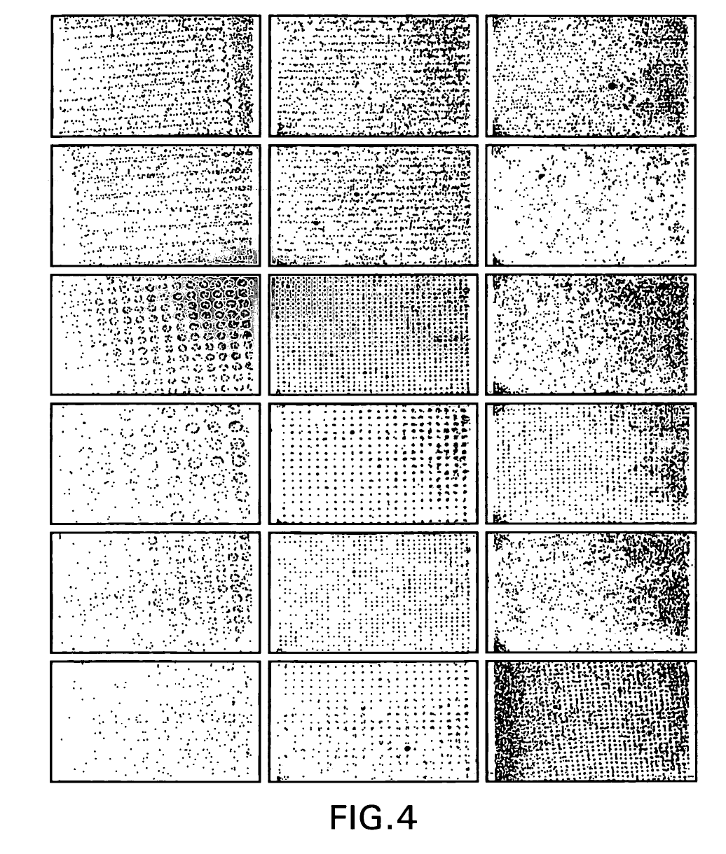

0.51 g of Fluorolink® (a perfluorinated polyether manufacture by Ausimont) were placed inside a stainless steel pressure vessel suitable for storing compressed carbon dioxide and adjusting the temperature and pressure thereof, such as that described, for example, by Hems, et al. (J. Mater. Chem., 1999, 9, 1403). A schematic representation of the vessel is shown in FIG. 1. The vessel was initially filled with supercritical CO₂ and the temperature adjusted to 35° C. and the pressure to 100 bar. The polymer dissolved in the supercritical CO₂ to form a colourless solution. The cell was then vented of the polymer solution via a capillary tube having an internal diameter of 127 μm and a length of either 10 cm or 30 cm while holding a patterned silicon wafer 3 cm away (10 cm capillary tube) or 2 cm away (30 cm capillary tube) from the tube. The solution exited the tube as a spray with a flow-rate of the solution was between 1 and 5 kg h⁻¹. The wafer (supplied by Mr Masaya Ishida of Seiko Epson) was patterned by lithography to give hydrophilic and hydrophobic areas, the polymer sticking to the hydrophobic areas. The wafer was then studied under the microscope. Photographs of the patterns obtained with the 10 cm capillary tube are shown in FIG. 4. The photographs show an identical polymer pattern to that on the original surface of the wafer. Similar results were obtained with the 30 cm capillary tube.

In Examples 1 to 3, the typical dot size of the deposited polymer was in the range 5 to 40 microns in diameter and the average space between the dots was 10 to 30 microns. This represents an excellent resolution level, showing the potential of the method of the present invention. Each of the squares in the photos is a single patterned area of the wafer and is 643×490 microns in size.

What is claimed is:

1. A method for the formation of a semiconductor device comprising:
dissolving or suspending a semiconducting material in a solvent phase comprising compressed carbon dioxide, the semiconducting material including at least one of a semiconducting small organic molecule and a semiconducting conjugated polymer;
depositing the solution or suspension onto a surface by means of a modified inkjet printing device; and
evaporating the solvent phase of the solution or suspension leaving a patterned semiconducting layer comprising the deposited semiconducting material.

2. A method according to claim 1, wherein:
the semiconducting layer is comprised of plural deposited regions of the semiconducting material;
a dimension extending across each deposited region is from 1 to 100 μm; and
a space between a pair of the plural deposited regions of the semiconducting material is from 10 to 30 μm.

3. A method according to claim 1, wherein the semiconducting device is an organic transistor.

4. A method according to claim 1, wherein the semiconducting device is an organic photovoltaic device.

5. A method according to claim 1, wherein the semiconducting device is an organic light emitting diode.

6. A method according to claim 1, wherein the depositing of the solution or suspension deposits the solution or suspension via a plurality of elongate bores.

7. A semiconducting layer in a device, the device comprising one of an organic transistor, an organic photovoltaic device and an organic light emitting diode, wherein the semiconducting layer has been formed by:
dissolving or suspending a semiconducting material in a solvent phase comprising compressed carbon dioxide, the semiconducting material including at least one of a semiconducting small organic molecule and a semiconducting conjugated polymer;
depositing the solution or suspension onto a surface of the device by means of a modified inkjet printing device; and
evaporating the solvent phase of the solution or suspension leaving the semiconducting layer comprising the deposited semiconducting material patterned by the modified inkjet printing device.

8. A semiconducting layer according to claim 7, wherein:
the semiconducting layer is comprised of plural deposited regions of the semiconducting material;
a dimension extending across each deposited region is from 1 to 100 μm; and
a space between a pair of the plural deposited regions is from 10 to 30 μm.

9. A semiconducting layer according to claim 7, wherein the semiconducting material comprises at least one of an emissive small organic molecule and an emissive conjugated polymer.

10. A semiconducting layer according to claim 9, wherein:
the semiconducting layer is comprised of plural deposited regions of the semiconducting material;
a dimension extending across each deposited region is from 1 to 100 μm; and
a space between a pair of the plural deposited regions is from 10 to 30 μm.

11. An array deposited on a surface, the array comprising a substance selected from DNA, proteins and catalysts, wherein the array includes a layer formed on the surface by:
dissolving or suspending the substance in a solvent phase comprising compressed carbon dioxide;
depositing the solution or suspension onto the surface by means of a modified inkjet printing device; and
evaporating the solvent phase of the solution or suspension leaving the substance patterned by the modified inkjet printing device.

12. An array according to claim 11, wherein:
the array is comprised of plural deposited regions of the substance;
a dimension extending across each deposited region is from 1 to 100 μm; and
a space between a pair of the plural deposited regions of the array is from 10 to 30 μm.
